(19) United States
(12) Patent Application Publication
(10) Pub. No.: US 2018/0146848 A1
(43) Pub. Date: May 31, 2018

(54) PEDIATRIC NASAL ENDOSCOPE, GASTROSCOPE AND AERODIGESTIVE SCOPE

(71) Applicant: The Regents of the University of Colorado, A Body Corporate, Denver, CO (US)

(72) Inventors: Joel FRIEDLANDER, Englewood, CO (US); Jeremy PRAGER, Englewood, CO (US); Emily DEBOER, Denver, CO (US); Robin DETERDING, Boulder, CO (US)

(21) Appl. No.: 15/850,939

(22) Filed: Dec. 21, 2017

Related U.S. Application Data
(63) Continuation of application No. PCT/US2016/039352, filed on Jun. 24, 2016.
(60) Provisional application No. 62/184,077, filed on Jun. 24, 2015.

(51) Int. Cl. A61B 1/273; A61B 1/07; A61B 1/018; A61B 10/04; A61B 1/005; A61B 1/06; A61B 1/05; A61B 1/12; A61B 1/015; A61B 1/233; A61B 1/267

(52) U.S. Cl. CPC A61B 1/2733; A61B 1/07; A61B 1/018; A61B 10/04; A61B 1/0052; A61B 1/0661; A61B 2503/06; A61B 1/12; A61B 1/015; A61B 1/051; A61B 1/233; A61B 1/2676; A61B 1/05

(57) ABSTRACT

TNE provides the opportunity to make the care of children with EoE and other gastrointestinal or aerodigestive conditions safer, more efficient, and less costly while simultaneously advancing our understanding of the pathophysiology and natural course of this condition. A pediatric endoscope was developed to facilitate TNE in children with EoE. The pediatric endoscope (combined gastroscope, bronchoscope, laryngoscope) includes a 3-4 mm flexible, fiber optic endoscope that allows HD TV viewing with the head of a pediatric bronchoscope that allows four way tip deflection, a scope stiffening apparatus to minimize the endoscopes flexibility when needed, a foot and hand activation to allow air/water insufflation and image/video capture, a light source, 2 mm biopsy channel.

### CROSS-REFERENCE TO RELATED APPLICATIONS

This application claims the benefit of U.S. Provisional Application No. 62/184,077, filed on Jun. 24, 2015.

### BACKGROUND OF THE INVENTION

### 1. Field of Invention

This invention relates to the evaluation and treatment of eosinophilic esophagitis, esophagitis, and aerodigestive conditions in children. More specifically, this invention relates to pediatric nasal endoscopes.

### 2. Brief Desciption of the Related Art

Eosinophilic esophagitis (EoE) is an increasingly common chronic inflammatory disease that affects children and adults with an estimated incidence of 1/10,000 in the United States. [Dellon E S, Gonsalves N, Hirano I, et al. ACG clinical guideline: Evidenced based approach to the diagnosis and management of esophageal eosinophilia and eosinophilic esophagitis (EoE). Am J Gastroenterol 2013; 108:679-92; quiz 693] Because of its potential to progress to esophageal stricture and the fact that symptoms do not always correlate with degree of eosinophilia, much attention has been paid to repeated assessment of the esophageal mucosa to insure mucosal healing following treatment. In contrast, the risks, cost and time commitment associated with traditional sedated esophagogastroduodenoscopy (EGD) can be significant and have raised concerns for providers and parents alike. [Gleich S J, Flick R, Hu D, et al. Neurodevelopment of children exposed to anesthesia: Design of the Mayo Anesthesia Safety in Kids (MASK) study. Contemp Clin Trials 2014; 41C:45-54] These dilemmas challenge the gastroenterologist to contemplate if EGD use in EoE is meeting the goal of Berwick's triple aim in health care to provide effective treatment, low cost care, and an optimal and safe healthcare experience. [Berwick D M, Nolan T W, Whittington J. The triple aim: care, health, and cost. Health Aff (Millwood) 2008; 27:759-69] Should EGD with biopsy be performed after each therapeutic change regardless of symptomatology, should EGD be reserved for patients who are not clinically responding to treatment, or should EGD not be performed again if patients are feeling well?

To address these questions, alternative methods are urgently needed to measure esophageal inflammation. While esophagoscopy with biopsies remains the gold standard technique for assessing mucosal inflammation, other technologies such as the Cytosponge, esophageal string test and confocal tethered endomicroscopy have emerged as potential alternatives. [Furuta G T, Kagalwalla A F, Lee J J, et al. The oesophageal string test: a novel, minimally invasive method measures mucosal inflammation in eosinophilic oesophagitis. Gut 2013; 62:1395-405; Tabatabaei N, Kang D, Wu T, et al. Tethered confocal endomicroscopy capsule for diagnosis and monitoring of eosinophilic esophagitis. Biomed Opt Express 2013; 5:197-207; Katzka D A, Geno D M, Ravi A, et al. Accuracy, safety, and tolerability of tissue collection by Cytosponge vs endoscopy for evaluation of eosinophilic esophagitis. Clin Gastroenterol Hepatol 2015; 13:77-83 e2.] To date, these tools, while less invasive, are still available only in research settings. [Dellon E S, Gonsalves N, Hirano I, et al. Am J Gastroenterol 2013; 108:679-92; quiz 693; Furuta G T, Kagalwalla A F, Lee J J, et al., Gut 2013; 62:1395-405]

Recent work has lead to the development of transnasal endoscopy/esophagoscopy (TNE) to assess the esophageal mucosa in adults. [Birkner B, Fritz N, Schatke W, et al. A prospective randomized comparison of unsedated ultrathin versus standard esophagogastroduodenoscopy in routine outpatient gastroenterology practice: does it work better through the nose? Endoscopy 2003; 35:647-51; Dumortier J, Josso C, Roman S, et al. Prospective evaluation of a new ultrathin one-plane bending videoendoscope for transnasal EGD: a comparative study on performance and tolerance. Gastrointest Endosc 2007; 66:13-9; Dumortier J, Ponchon T, Scoazec J Y, et al. Prospective evaluation of transnasal esophagogastroduodenoscopy: feasibility and study on performance and tolerance. Gastrointest Endosc 1999; 49:285-91; Hu C T. Gauze pledgetting versus endoscopic-guided aerosolized spray for nasal anesthesia before transnasal EGD: a prospective, randomized study. Gastrointest Endosc 2010; 71:11-20; Mokhashi M S, Wildi S M, Glenn T F, et al. A prospective, blinded study of diagnostic esophagoscopy with a superthin, stand-alone, battery-powered esophagoscope. Am J Gastroenterol 2003; 98:2383-9; Mulcahy H E, Riches A, Kiely M, et al. A prospective controlled trial of an ultrathin versus a conventional endoscope in unsedated upper gastrointestinal endoscopy. Endoscopy 2001; 33:311-6; Yagi J, Adachi K, Arima N, et al. A prospective randomized comparative study on the safety and tolerability of transnasal esophagogastroduodenoscopy. Endoscopy 2005; 37:1226-31] In contrast to traditional EGDs, TNE offers advantages including that it can be performed in an outpatient clinic room, requires no anesthesia or sedation, uses an adult transnasal gastroscope that is tolerated by adults and procures samples adequate for assessment of Barrett's Esophagus. [Shariff M K, Bird-Lieberman E L, O'Donovan M, et al. Randomized crossover study comparing efficacy of transnasal endoscopy with that of standard endoscopy to detect Barrett's esophagus. Gastrointest Endosc 2012; 75:954-61; Saeian K, Staff D M, Vasilopoulos S, et al. Unsedated transnasal endoscopy accurately detects Barrett's metaplasia and dysplasia. Gastrointest Endosc 2002; 56:472-8] However, the endoscopes used in the adult procedures are not appropriate for use in pediatric setting. Adult endoscopes have a large bulky head, will not fit in many pediatric size nasal passages, do not have stiffening capability for improve maneuvariabilty, do not have foot controls, are not able to be used in pediatrics, and are unable to be used for bronchoscopy. Accordingly, what is needed is a device and associated methodology that can be used to adapt TNE to assess the esophageal mucosa, gastric, and duodenal, tracheal, and bronchial mucosa in children. The present invention provides tools and techniques to meet this important need.

### BRIEF SUMMARY OF THE INVENTION

The long-standing but heretofore unfulfilled need for is now met by a new, useful, and nonobvious pediatric nasal endoscope. The invention includes a miniaturized 3-4 mm flexible, fiber optic endoscope approximately 1 meter in length that allows high resolution, high definition, and clear optics of the nasal mucosa, pharynx and upper gastrointestinal tract with the small head of a pediatric bronchoscope that allows four way tip deflection to allow use by individuals with small hand sizes, a foot pedal to allow air/water insufflation for ease of use, a bright light source, a scope stiffening apparatus that will allow utilization in aerodigestive medicine (combined ENT-laryngoscopy, Pulmonary (bronchoscopy), and Gastroenterology (EGD) Procedures) and a 2 mm biopsy channel to assure utilization of currently available endoscopic tools.

Unsedated laryngoscopy in pediatric otolaryngology and pediatric pulmonology has been performed in pediatric patients. [Wood R E. Evaluation of the upper airway in children. Curr Opin Pediatr 2008; 20:266-71] We hypothesized that TNE could be adapted to provide a safe and effective tool to monitor and sample the mucosa of children with EoE. Ultra-slim flexible endoscopes were developed to create instruments that could be tolerated by children, while still allowing for the removal of adequate samples. This scope design is unique in that it can be used in pediatric nasal endoscopy, pediatric nasal bronchoscopy, and pediatric laryngoscopy. This design differs from current adult nasal endoscopes in production in terms of numerous aspects including that that it is narrower, lighter, clearer, has foot controls, has smaller accessible hand controls to control the tip and be easier to maneuever. This scope will also have a stiffening capability and narrow tip to allow it to be used in aerodigestive medicine and other medical and surgical specialties. The present disclosure documents the performance of TNE with biopsies using these ultra-slim flexible endoscopes to assess the esophageal, gastric, duodenal, tracheal, and bronchial mucosa in pediatric subjects with EoE. The performance was assessed in part through the evaluation of parental and patient subject responses to TNE, the assessment of the ability to procure samples that would be adequate to monitor disease, monitoring adverse events, and recording procedure duration and charges generated. This assessment showed that unsedated transnasal endoscopy using the pediatric nasal endoscope disclosed herein offers an excellent alternative to sedated esophagogastroduodenoscopy.

Unsedated transnasal endoscopy (TNE) in adults is safer and less costly than sedated esophagogastroduodenoscopy (EGD). TNE with biopsies can be adapted as an effective tool to monitor the esophageal, gastric, and duodenal mucosa of children with eosinophilic esophagitis (EoE) or other conditions of the upper gastrointestinal tract with the proper tools and techniques. This technique can dramatically increase the safety and decrease cost in the care of children. The present disclosure documents the development of the performance of TNE with biopsies in pediatric EoE.

Subjects between 8 and 17 years of age with EoE, and their parents, were enrolled in the study. Unsedated TNE was performed. The currently available smaller endoscopes designed for bronchoscopy as a 2.8 mm (1.2 mm channel) or a 4 mm flexible bronchoscope (2 mm channel) were used, and esophageal biopsies were procured. These scopes were shorter than our currently proposed pediatric nasal endoscope and were without water channels, suction, air, foot control, high definition optics, or stiffening capability. Biopsy analysis, duration, adverse events, and billing charges of TNE were assessed. Immediately after TNE and a minimum of 2 weeks later, the mGHAA-9 (modified Group Health Association of America) and a preference questionnaire were completed, respectively.

Twenty-one of 22 enrolled subjects completed TNE. TNE was tolerated with no significant adverse events. Histopathological analysis revealed 0 eos/hpf (n=12), <15 eos/hpf (n=4), and >15 eos/hpf (n=5) and total epithelial surface area of mucosal biopsies samples from either TNE forceps compared to those obtained with standard endoscopic forceps was not statistically different. All parents and 76.2% of subjects would undergo the TNE again. TNE was preferred over EGD by 85.7% of parents and 52.4% of subjects. mGHAA-9 revealed a high degree of satisfaction (average 43.19+/−2.6 maximum score-45). Charges associated with TNE were 60.1% less than previous EGDs. The results of this study show that unsedated TNE is a preferred, efficacious, and lower cost procedure when monitoring esophageal mucosa of children with EoE.

In a first aspect the present invention provides an endoscope for assessment of the esophageal mucosa in children. The endoscope can have a flexible endoscope shaft having a first end, a second end, a length of about 0.8 meters to about 1.3 meters (preferably about 0.9 meters to about 1.2 meters, more preferably about 1.0 meters to about 1.1 meters, or about 1.05 meters), an outer diameter of between about 3.0 mm to about 4.0 mm (preferably about 3.0 mm to about 5.0 mm, more preferably about 3.25 mm to about 4.0 mm, about 3.5 mm to about 4.0 mm, about 3.5 mm, or about 4.0 mm) and having an inner channel lumen of about 1.5 mm to about 2.5 mm in diameter (preferably about 1.75 mm to about 2.25 mm, or about 2.0 mm). The lumen extends substantially the length of the shaft, and will generally have an opening at the distal-most portion of the second end to allow a surgical instrument to partially exit the lumen for placement of the tool in proximity to a tissue of interest. The shaft can be configured to facilitate irrigation and suction at the second end of the shaft, such as by including connection to a source for an irrigation liquid and/or suction and passage across the shaft of the endoscope for the liquid and its return. The endoscope according to the first aspect has a handle disposed at first end of the shaft. The handle can have a single or dual control to adjust the disposition of the second end of the shaft. The control enables four-way tip deflection of the second end of the shaft. This allows a user to direct the distal end of the shaft to facilitate visualization and sampling of desired tissues at the distal or second end. The endoscope according to the first aspect has an image sensor at the second end of the shaft. The image sensor facilitates imaging of tissues at the distal end of the endoscope when the endoscope is inserted within a cavity of a subject. The endoscope according to the first aspect has a light source disposed at the second end of the shaft to illuminate the area surrounding the distal end of the shaft.

In an advantageous embodiment the endoscope according to the first aspect can have a foot pedal or hand control to actuate suction or irrigation of the endoscope. The control can be integral to the handle to actuate suction or irrigation of the endoscope. In further embodiments the endoscope according to the first aspect can have a camera to facilitate visualization within the cavity of the subject. The image sensor can be a charge-coupled device (CCD) sensor, a complementary metal-oxide-semiconductor (CMOS) sensor, N-type metal-oxide-semiconductor (NMOS) sensor or a high definition video chip.

In an advantageous embodiment the endoscope according to the first aspect can have a scope shaft stiffening component. The scope shaft stiffening component can be used to selectively reduce the flexibility of the scope shaft. In other words, a user can selectively alter the stiffness of the shaft during use to suit the particular stiffness needed to execute a procedure or direct the placement of the shaft. The scope shaft stiffening component can adapted to facilitate the use of the endoscope in aerodigestive medicine. Additionally, the lumen can have an opening at the distal-most end of the second end of the shaft. This allows for the passage of instruments and for the irrigation and suction of biological tissues through the length of the shaft and their partial exit from the shaft.

In a second aspect the present invention provides an endoscope for transnasal endoscopy in children. The endoscope according to the second aspect has a flexible endoscope shaft having a first end and a second end and has a diameter dimensioned for insertion into the nasal cavity of a child, a length of about 0.8 meters to 1.2 meters, and has an inner channel lumen configured to receive an elongate surgical instrument, the lumen extending substantially the length of the shaft. The shaft is configured to facilitate irrigation and suction at the second end of the shaft. The endoscope according to the second aspect has a handle disposed at first end of the shaft, the handle including a single or dual control to adjust the disposition of the second end of the shaft thereby enabling four-way tip deflection. The endoscope according to the second aspect also has an image sensor at the second end of the shaft to facilitate imaging at the distal end of the endoscope when the endoscope is inserted within the nasal cavity of a subject. Lastly, the endoscope according to the second aspect has a light source disposed at the second end of the shaft to illuminate the area surrounding the distal end of the shaft.

In an advantageous embodiment the endoscope according to the second aspect can have a foot pedal or hand control to actuate suction or irrigation of the endoscope. The control can be integral to the handle to actuate suction or irrigation of the endoscope. In further embodiments the endoscope according to the second aspect can have a camera to facilitate visualization within the cavity of the subject. The image sensor can be a charge-coupled device (CCD) sensor, a complementary metal-oxide-semiconductor (CMOS) sensor, N-type metal-oxide-semiconductor (NMOS) sensor or a high definition video chip.

In an advantageous embodiment the endoscope according to the second aspect can have a scope shaft stiffening component. The scope shaft stiffening component can adapted to facilitate the use of the endoscope in aerodigestive medicine. Additionally, the lumen can have an opening at the distal-most end of the second end of the shaft. This allows for the passage of instruments through the length of the shaft and their partial exit from the shaft.

In a third aspect the present invention provides a second endoscope for transnasal endoscopy in children. The endoscope according to the third aspect has a flexible endoscope shaft having a first end, a second end, a shaft diameter dimensioned for insertion into the nasal cavity of a child, a shaft length adapted to facilitate insertion of the shaft through the nasal cavity to the esophageal mucosa of a child, and an inner channel lumen configured to receive an elongate surgical instrument. The lumen extends substantially the length of the shaft. The shaft is further configured to facilitate irrigation and suction at the second end of the shaft. The endoscope according to the third aspect has a handle disposed at first end of the shaft. The handle has a single or dual control to adjust the disposition of the second end of the shaft thereby enabling four-way tip deflection. The endoscope according to the third aspect also has an image sensor at the second end of the shaft to facilitate imaging at the distal end of the endoscope when the endoscope is inserted within the nasal cavity of a subject. In addition, the endoscope according to the third aspect has a light source disposed at the second end of the shaft to illuminate the area surrounding the distal end of the shaft.

In an advantageous embodiment the endoscope according to the third aspect can have a foot pedal or hand control to actuate suction or irrigation of the endoscope. The control can be integral to the handle to actuate suction or irrigation of the endoscope. In further embodiments the endoscope according to the third aspect can have a camera to facilitate visualization within the cavity of the subject. The image sensor can be a charge-coupled device (CCD) sensor, a complementary metal-oxide-semiconductor (CMOS) sensor, N-type metal-oxide-semiconductor (NMOS) sensor or a high definition video chip.

In an advantageous embodiment the endoscope according to the third aspect can have a scope shaft stiffening component. The scope shaft stiffening component can adapted to facilitate the use of the endoscope in aerodigestive medicine. Additionally, the lumen can have an opening at the distal-most end of the second end of the shaft. This allows for the passage of instruments through the length of the shaft and their partial exit from the shaft.

### BRIEF DESCRIPTION OF THE DRAWINGS

For a fuller understanding of the invention, reference should be made to the following detailed description, taken in connection with the accompanying drawings, in which:

FIG. 1 is an image showing a biopsy with active EoE using a standard 2.8 mm EGD forceps. The surface area is 0.10 mm².

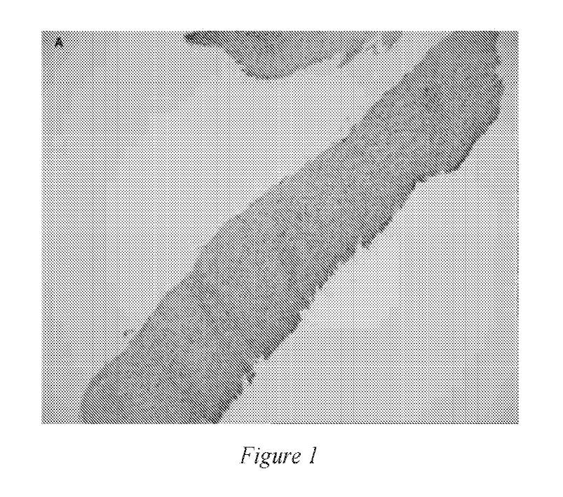

FIG. 2 is an image showing a biopsy from the same patient with active EoE using TNE 1.2 mm forceps. The surface area is 0.12 mm².

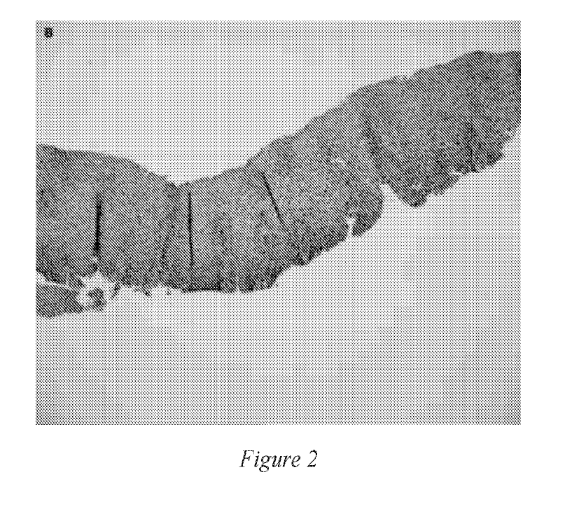

FIG. 3 is an image taken from a subject with active furrowing and eosinophilic exudates.

FIG. 4 is a drawing depicting a pediatric nasal endoscope according to aspects of the present invention.

FIG. 5 is a drawing depicting a pediatric nasal endoscope biopsy forceps according to aspects of the present invention.

### DETAILED DESCRIPTION OF THE PREFERRED EMBODIMENT

The emergence of EoE has led to a renewed interest in determining pathogenic mechanisms of esophageal inflammation and sampling of the esophageal mucosa to assess for mucosal healing. Despite the rapid progress in establishing diagnostic criteria, treatments, and novel genes related to pathogenic mechanisms that can significantly impact EoE patients, limited data is available to document the natural history of EoE. This lack of understanding has led to the present clinical practice of multiple, high-cost, and higher-risk sedated assessments of the esophageal mucosa to ascertain whether eosinophilia has resolved following treatment. If eosinophilia resolves, a predicate determination is made that the likelihood for EoE-related complications is diminished. If eosinophilia persists, efforts are made to resolve inflammation regardless of symptomatology, with its subsequent impact on quality of life and costs of care. In this regard, novel devices and sampling methodologies are urgently needed. To address this and offer a new tool in the evaluation of EoE, we sought to determine if TNE could sample the esophageal mucosa in a way that was well-tolerated and adequate. In light of the emergent need for more efficient methods of esophageal mucosal evaluation in EoE, we performed this study within the confines of a multi-disciplinary team to perform TNE with biopsies in a pediatric population. We chose this population because of the urgent need to minimize the repetitive risks of anesthesia, improve the understanding of EoE pathogenesis, and to ultimately identify novel therapeutic targets.

Unsedated TNE is an established technique in a number of pediatric and adult subspecialties, but it has not been used by pediatric gastroenterologists. [Birkner B, Fritz N, Schatke W, et al. Endoscopy 2003; 35:647-51; Dumortier J, Josso C, Roman S, et al. Gastrointest Endosc 2007; 66:13-9; Dumortier J, Ponchon T, Scoazec J Y, et al. Gastrointest Endosc 1999; 49:285-91; Hu C T. Gastrointest Endosc 2010; 71:11-20; Mokhashi M S, Wildi S M, Glenn T F, et al. Am J Gastroenterol 2003; 98:2383-9; Mulcahy H E, Riches A, Kiely M, et al. Endoscopy 2001; 33:311-6; Yagi J, Adachi K, Arima N, et al. Endoscopy 2005; 37:1226-31] A number of studies have described the advantages, limitations, and challenges of TNE use, and in 2010, the America Society of Gastrointestinal Endoscopy developed a guideline for the use of TNE in adults. [Committee A T, Rodriguez S A, Banerjee S, et al. Ultrathin endoscopes. Gastrointest Endosc 2010; 71:893-8; Faulx A L, Catanzaro A, Zyzanski S, et al. Patient tolerance and acceptance of unsedated ultrathin esophagoscopy. Gastrointest Endosc 2002; 55:620-3; Faulx A L, Vela S, Das A, et al. The changing landscape of practice patterns regarding unsedated endoscopy and propofol use: a national Web survey. Gastrointest Endosc 2005; 62:9-15; Tatsumi Y, Harada A, Matsumoto T, et al. Current status and evaluation of transnasal esophagogastroduodenoscopy. Dig Endosc 2009; 21:141-6] This guideline increased attention to cost containment, and the recent upswing in interest in esophageal diseases led to renewed interest in this technique. [Faulx A L, Catanzaro A, Zyzanski S, et al. Gastrointest Endosc 2002; 55:620-3; Chak A, Alashkar B M, Isenberg G A, et al. Comparative acceptability of transnasal esophagoscopy and esophageal capsule esophagoscopy: a randomized, controlled trial in veterans. Gastrointest Endosc 2014; 80:774-82; Lin L F, Shen H C. Unsedated transnasal percutaneous endoscopic gastrostomy carried out by a single physician. Dig Endosc 2013; 25:130-5; Cho S, Arya N, Swan K, et al. Unsedated transnasal endoscopy: a Canadian experience in daily practice. Can J Gastroenterol 2008; 22:243-6] A recent study also demonstrated the utility of TNE in adult's with Barrett's esophagus. [Tatsumi Y, Harada A, Matsumoto T, et al. Current status and evaluation of transnasal esophagogastroduodenoscopy. Dig Endosc 2009; 21:141-6; Chak A, Alashkar B M, Isenberg G A, et al. Comparative acceptability of transnasal esophagoscopy and esophageal capsule esophagoscopy: a randomized, controlled trial in veterans. Gastrointest Endosc 2014; 80:774-82; Bush C M, Postma G N. Transnasal esophagoscopy. Otolaryngol Clin North Am 2013; 46:41-52]. To date, only one study evaluated unsedated trans oral endoscopy in children and concluded that it improved time and safety in assessing 21 children for evaluation of abdominal pain, dyspepsia, and dysphagia. [Bishop P R, Nowicki M J, May W L, et al. Unsedated upper endoscopy in children. Gastrointest Endosc 2002; 55:624-30]

With the rapidly increasing prevalence of EoE, limited knowledge regarding its pathophysiology, and emerging clinical needs to assess the esophageal mucosa, we sought to determine whether TNE in pediatric EoE would be a feasible and efficacious tool. Results of our study reveal that patients and parents experienced with sedated EGD tolerate TNE well, and that patients and their parents prefer TNE compared to EGD. It is likely that the limited side effect profile and complete lack of serious adverse events contributed to the finding that 52.4% of child subjects (4 subjects preferring neither EGD or TNE) and the 85.7% of parents (1 parent preferring neither TNE or EGD) preferred unsedated TNE to sedated EGD. In that parents often make decisions about procedures in pediatrics and a majority of children prefer the procedure, these percentages are indicative of a highly successful alternative to EGD. Immediate benefits of this preference for patients include improved patient satisfaction and increased safety by eliminating anesthesia.

In many ways our results are quite similar to that reported in adult studies. For example, a large Canadian Study by Cho et al. evaluating 231 patients with an average age of 57 years for routine TNE; their study also found that TNE was well tolerated, safe and feasible. [Cho S, Arya N, Swan K, et al. Unsedated transnasal endoscopy: a Canadian experience in daily practice. Can J Gastroenterol 2008; 22:243-6] This study was different, however, in that the patients were primarily adults, the scope used was larger (5.3 mm) and duodenal intubation was performed. Our study evaluated the use of 2 smaller endoscopes, smaller biopsy forceps, and TNE performance in children. Some areas of divergence between our findings and similar adult studies include that (1) in our study both the parents and child subjects evaluated the technique, (2) the adequacy of smaller forceps to evaluate the esophageal mucosa in EoE was assessed, and (3) the actual rather than contemplative type of future endoscopy preferred by subjects who have undergone multiple previous EGD's was examined. These findings augment the results of this research and its potential application to adult and pediatric endoscopy practices.

We are particularly encouraged by our findings for several reasons. First, there was great interest in this procedure amongst patients and parents. We only needed to screen 22 subjects to enroll the 21 subjects reported here. This is likely explained by the fact that the EoE patient population represents a very engaged, experienced, and educated population that is readily seeking alternative methods. We are highly confident that this is a technically feasible procedure, and are further encouraged by its overall rapid success that was facilitated by a multidisciplinary pediatric team dedicated to the care of children with aerodigestive diseases and EoE. Our study provides strong support for larger studies to validate this approach that will provide novel insights into the natural history of EoE and significantly improve the lives of children with EoE in a safer, cost effective, and efficacious manner.

Second, our study found a high level of satisfaction and enthusiasm to repeat the TNE. The overwhelming majority of patients and parents were satisfied and preferred unsedated TNE compared to standard EGD. Subject responses in the qualitative survey identified critical elements including the lack of anesthesia, the presence of parents during the procedure, the limited duration of the procedure and rapid recovery. TNE was safe, as evidenced by the fact that no significant adverse events or event needed subsequent treatment or evaluation. The subjects and parents appreciated the improvement in their quality of life with TNE, as it allowed them the ability to return to school and work and eat shortly afterwards. In fact, several families noted the patients returned to school or a sport activity after the TNE. The time at CHCO for a standard EGD is 3 hours compared to 60-90 minutes for the TNE, a time that included not only the TNE but also research protocol documentation. This 3-hour procedure center time for EGD usually includes check in, pre-operative evaluation by nursing, gastroenterology, and anesthesia, the procedure itself, recovery, and discharge instructions. The 60-90 minute time for TNE in clinic included research documentation, pre-procedural documentation, the procedure itself, and discharge instructions. Most of these improvements in time reduction and increased satisfaction, noted above, are related to the effects of eliminating anesthesia or sedation for TNE. Not only does this practice seem to improve satisfaction of patients and parents, but there is also a significant likelihood it decreases the risk of adverse medication reactions, aspiration, and possible effects on the developing pediatric brain. [Gleich S J, Flick R, Hu D, et al. Contemp Clin Trials 2014; 41C:45-54] This is an emerging concern amongst pediatric anesthesiologists. While pediatric subjects without sedation or general anesthesia noted a mild sore throat and gagging, this was minor enough that the majority chose follow up TNE for their EoE evaluation after their initial study procedure. This has been confirmed as more than a hypothetical question, with several of our subjects requesting follow up TNE after the study concluded.

The third positive outcome of our study relates to the integrity of the mucosal sample. Regarding the techniques effectiveness in evaluating mucosal esophageal sample, we found that the epithelial surface area needed for eosinophil count evaluation was not significantly different from the standard EGD 2.8 mm biopsy forceps compared to either of the TNE 2 mm or 1.2 mm biopsy forceps. This finding provides a high level of confidence that the sample procured at the time of TNE will have the same surface area compared to that obtained with the gold standard EGD biopsy forceps. The 2 mm forceps were also able to procure lamina propria.

The final areas of interest in this study were the reduction in cost and increase in efficiency. Financial benefits of TNE include the fact that TNE incurred fewer charges and required less time away from work and school when compared to a standard sedated pediatric EGD. The project demonstrated a significant 60.1% drop in charges. The majority of this reduction in cost is related to the lack of anesthetic/anesthesiologist during TNE. The significance of this cannot be understated. For example, if our institution were to perform 100 sedated EGD's per year for EoE at a hypothetical average, non-insurance adjusted charge of $9,390 dollars per general anesthesia provided endoscopy encounter, this would accumulate $939,000 total charges per year for EoE. This would include all facility charges, physician, pathology and anesthesia fees. If these 100 EGDs for EoE were converted to unsedated TNE, this could translate to a healthcare systems charge savings of $564,000 dollars per year. These are possible charges however and not respective insurance rates.

Several areas will be addressed in future evaluations. First, for practical reasons, we needed to use 2 different sized endoscopes for TNE. Future work will standardize this for patient comfort and biopsy size. Second, although the 1.2 mm forceps were not able to procure lamina propria, the 2 mm forceps in this study were able to obtain lamina propria. While this section of the tissue has been used to grade fibrosis, this metric has not been standardized or become a gold standard for clinical assessments. Our study demonstrates feasibility in pediatrics, but evaluating a much larger cohort is required to achieve a significant power for safety and other metrics. Power analysis based on our own institution's quality and safety data would necessitate over 10,000 endoscopies to find a single significant adverse event. This could be remedied with the development of further databases, as this technique is increasingly used at our institution or in a national program evaluating its use in pediatrics. Finally, we undertook this study in a multi-disciplinary collaboration with our pediatric pulmonary and otolaryngology colleagues. This was done for study design to maximize patient comfort during TNE development in pediatrics, a strong interest in aerodigestive and eosinophilic disorders by gastroenterologists, otolaryngologists, and pulmonologists alike, and the need for a more pragmatic multi-disciplinary approach to diagnosing and managing EoE as it presents in different single specialty clinics. Since the study initiation, the gastroenterologist, otolaryngologist, and pulmonologists have been trained in single physician TNE with biopsies that further improves cost and efficiency, diagnosis, and referral for management of EoE.

In conclusion, the implementation of TNE in pediatric gastroenterology for the evaluation of pediatric EoE is safe, preferred by patients and parents alike, and has the potential to dramatically reduce costs. Thus it appears that TNE would be measured as a highly effective practice in pediatric EoE management per Berwick's description of the triple aim: the pursuit of improved experience of care, the health of populations should be improved, and the cost of per capita healthcare should be decreased. [Berwick D M, Nolan T W, Whittington J. Health Aff (Millwood) 2008; 27:759-69] This suggests that TNE use should be highly considered as an alternative to standard sedated EGD or esophagoscopy for the follow up evaluation of pediatric EoE. The technique will continue to be refined and improved, offering more opportunities for its use in monitoring response to therapeutics, obtaining follow-up evaluations, and performing research in EoE.

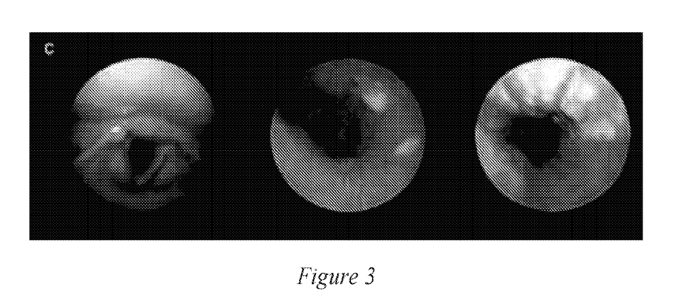

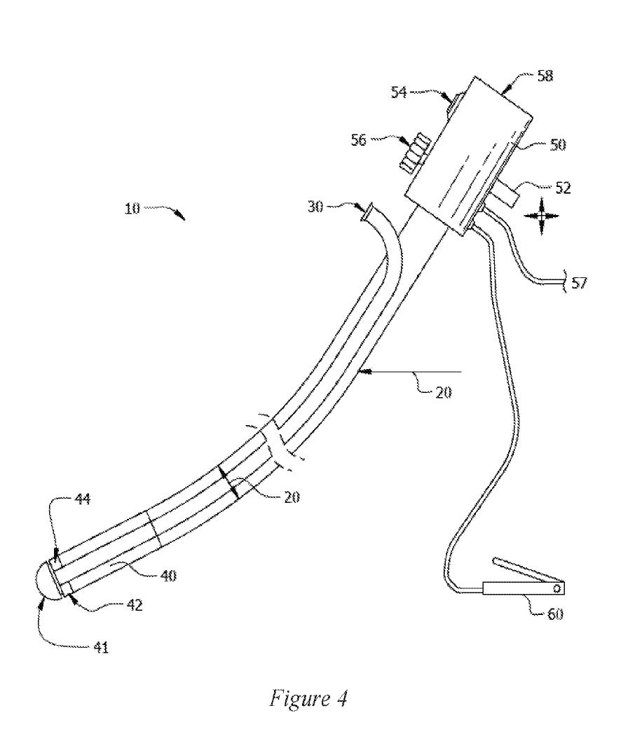

Turning to FIG. 4, a pediatric nasal endoscope 10 was developed to perform TNE in children. The pediatric nasal endoscope 10 includes a flexible endoscope shaft 20 constructed from medical-grade slippery (such as a hydrophobic) material with a slick coating having a length of about 1.05 meters and a width of about 3.5 mm. The flexible endoscope shaft 20 has a biopsy channel 30 running the length of the endoscope shaft and is adapted to slidingly receive a pediatric nasal endoscope biopsy forceps 70 (See FIG. 3) within the lumen of the channel or allow suction or irrigation 30. The distal end 40 of the flexible endoscope shaft 20 is rounded and can be flat or if the end user wishes designed to be terminated with an optional, removable soft silicone tip 41. The distal end 40 of the flexible endoscope shaft 20 also includes a high lumen LED 42 to provide light at the tip and a high-resolution video capture device to capture images or video in the region of the distal end 40 of the flexible endoscope 20. The proximal end 50 of the flexible endoscope shaft 20 can include a single 4-way tip deflection control lever 52 to control the displacement of the endoscope's tip, a button 54 to actuate photo or video capabilities of the endoscope, a hand control 56 to operate air and/or water suction, a line out 57 to a imaging system such as a computer monitor, an optional scope stiffening device 58 to allow its use in aerodigestive medicine. A foot pedal 60 can also be coupled to the endoscope to activate and control water flow and air suction.

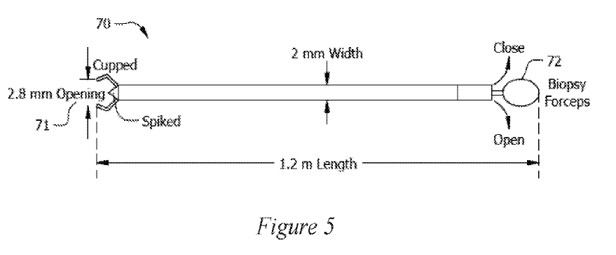

As discussed above, the flexible endoscope shaft 20 has a biopsy channel 30 running the length of the endoscope and is adapted to slidingly receive a pediatric nasal endoscope biopsy forceps 70 (See FIG. 5) within the lumen of the channel 30. Turning to FIG. 5, an exemplary pediatric nasal endoscope biopsy forceps 70 is illustrated. The pediatric nasal endoscope biopsy forceps 70 has a length of about 1.2 meters, which is slightly longer than the length of the biopsy channel 30, a width of about 2 mm, and opposing ends forming a distal end 71 and a proximal end 72. The distal end 71 includes a cupped and spiked tip with an opening of about 2.8 mm to 5 mm when fully open. The proximal end 72 includes an actuator to open and close the tip at the distal end 71 of the forceps 70. Examples 1 & 2, presented below, document the development of the transnasal endoscopy/esophagoscopy (TNE) to assess the esophageal mucosa in children using the pediatric nasal endoscope.

### EXAMPLE 1

### Materials and Methods

Subjects ages 8-17 years of age between March 2014 and January 2015 with a defined diagnosis of EoE and who had undergone at least one prior EGD under anesthesia were recruited from the outpatient clinic at Children's Hospital Colorado (CHCO). At the time of the scheduled clinically indicated follow up appointment, subjects were approached if their primary GI provider felt a follow-up esophagoscopy was needed to evaluate their clinical response to therapy. Subjects were queried if they would be interested in having an unsedated TNE with movie distraction performed instead of a sedated EGD. If so, informed consent was obtained and demographic data collected.

Subjects were instructed to not eat or drink for 2 hours prior to the TNE. In a standard clinic room, subjects were asked to sit in a chair designed for outpatient laryngoscopic procedures. Two to six sprays of 4% aerosolized lidocaine were applied to the nares to achieve topical anesthesia. Subject distraction was accomplished using either HMZ-T3W 3D movie goggles (Sony Corporation, Tokyo, Japan) or Cinemizer Goggles (Carl Zeiss AG, Oberkochen, Germany) dependent on facial size to facilitate viewing an immersive movie or television program of their choice. Parents remained in the room for the duration of the study. For study design purposes and patient comfort one of two designated pulmonologists (ED, RD) or a single otolaryngologist (JP) performed transnasal laryngoscopy using an Olympus BFXP160F 2.8 mm bronchoscope (1.2 mm biopsy channel) in 11/21 subjects and 10/21 subjects using a 4 mm BPMP160F (2 mm biopsy channel) ending with the endoscope in the proximal esophagus. The gastroenterologist (JF) performed esophagoscopy and biopsy collection (3 from proximal and 3 from distal esophagus). Visual confirmation of the adequacy of the biopsy specimens was performed before withdrawing scope. Adverse events, subject symptoms, duration of TNE in 5 minute intervals up to 15 minutes were collected. After the procedure, families were asked to answer the mGHAA-9 (modified Group Health Association of America) endoscopy satisfaction questionnaire and discharged home.

Subjects were called the evening of the procedure and >72 hours later to evaluate for any adverse events. A minimum of two weeks but not greater than 10 weeks after TNE, the subjects and parents were asked to answer an electronic qualitative survey regarding their experience with TNE.

A single pediatric pathologist (KC) evaluated biopsy specimens to assess for size of the sample and inflammatory findings including eosinophil enumeration. To assure adequate high power field (hpf) analysis, the total epithelial surface area used to count eosinophils was analyzed using graphical software and analysis (cellSens Standard, 2013, Olympus America, USA). This was accomplished by comparing the subject's available previous esophageal biopsies using a standard 2.8 mm biopsy forceps to the 1.2 or 2 mm biopsy forceps specimens that were collected during TNE.

Charges from TNEs and subjects' previous isolated sedated EGD were collected to compare the cost of the two procedures. Subjects who underwent combined procedures that may have prolonged sedation such as pH probes, pH impedance probes, colonoscopy, or flexible sigmoidoscopy at their previous endoscopy were excluded from this calculation (n=12). University of Colorado Institutional Review Board (COMIRB-13-2721) approved all study procedures.

Data was recorded into a Red-Cap Secure Database. It is reported as qualitative measure as noted with average, mean, and standard deviation (SD). Surface area analysis to assure adequate specimen size was performed using student's paired, non-parametric, t-test. Charge analysis was performed using unpaired t-test.

### EXAMPLE 2

### Evaluation of the Pediatric Nasal Endoscope

Of 22 subjects referred for endoscopy, 22 were contacted and 21 subjects (95.5%) enrolled in this study. One female subject chose not to participate because of “sensory issues.” Clinical features of these 21 subjects are shown in Table 1. The average age was 13.04 yrs (+\−2.7 yrs SD, range from 8-17 years). Subject numbers 1, and 13-21 underwent TNE using the 4 mm endoscope and were aged ranging from 8-16 years. Subject numbers 2-12 underwent TNE using the 2.8 mm endoscope and were aged ranging from 10-17 years. The average number of endoscopies previously performed on the subject cohort was 2.19 (SD+/−1.12). All subjects tolerated TNE with no significant adverse events. Duration of TNE procedures decreased as the endoscopists (JF, ED, JP, RD) became more experienced with TNE. (Table 2). The youngest child was 8 years old and was able to tolerate the 4 mm endoscope without difficulty. Symptoms associated with the TNE included gagging and sore throat (Table 3). No adverse event was associated with any emergency department evaluation or unintended evaluation or treatment. One subject had a panic attack prior to the procedure but was still able to complete the TNE without any additional medication. She had a previous history of an anxiety disorder.

Post-procedure assessment revealed a high degree of satisfaction and comfort with the TNE immediately after and at subsequent survey. mGHAA-9 satisfaction instrument average score was 43.19+/−2.6 n=21; maximum 45. A high percentage of subjects reported satisfaction with TNE, child subjects (81%) and parents (90.5%). This is as compared to 81% of combined parent/child subjects satisfied with their previous sedated EGD when asked about it at time of TNE survey. Subjects expressed greater concerns for EGD than TNE on qualitative instrument (61.9% vs. 28.6% respectively). The majority of children (76.2%) would repeat TNE and 100% of parental subjects were willing to have their child undergo the procedure again. More than half of child subjects 52.4% preferred TNE, with 4 subjects not preferring either TNE or sedated EGD, while 85.7% of parental subjects preferred TNE for their child. (Table 4). Reasons for parental preference of TNE included: no anesthesia (61.9%) faster procedure and recovery (52.3%), parental presence during the procedure (28.5%), and lower cost (19%).

Visual TNE findings revealed 11 subjects with normal esophagoscopy, 9 with furrowing and one with furrowing and exudates. Visual findings correlated to the appropriate histologic findings in 85.7% of subjects. In those subjects where visual and histological findings did not correlate, 2 subjects with visual furrowing had normal biopsies, and one with normal appearing mucosa showed histological evidence of eosinophilia <15 eos hpf. (Image 1,Table 5)

Biopsy specimens revealed 12 normal biopsies, 4 with less than 15 eosinophils per hpf, and 5 with greater than 15 eosinophils per hpf. (Table 5, Image 1) No significant difference was identified when comparing total epithelial surface area of TNE biopsies to the biopsy surface area of the matched subject's previous EGD. (Table 5) One subject that was initially evaluated at an outside institution did not have his previous biopsies available for analysis. Total epithelial surface area of mucosal biopsies samples from TNE forceps compared to those obtained with standard endoscopic forceps was not statistically different. (0.33 mm²+/−0.09 vs. 0.38 mm²+/−0.14 mm; p=0.308; n=11; TNE 1.2 mm forceps vs EGD 2.8 mm forceps+/−SD 0.50 mm2+/−0.15 vs. 0.52 mm2+/−0.19; p=0.496, n=9; TNE 2 mm forceps vs EGD 2.8 mm forceps+/−SD). Although there appears to be a surface area difference between the two 2.8 mm control groups (0.38 mm2 and 0.52 mm2), sub-analysis revealed no significant difference was present using unpaired, non-parametric t-test. (p>0.05)

Of the 21 subjects who underwent TNE, 11 had charge data that was comparable and available for analysis. Charges for TNE were calculated to be 60.1% less than sedated EGD with biopsies, including anesthesia, pathology, facility fees, and physician fees.

All references died in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described.

TABLE 1
Demographics

| | | | Average Number |
|---|---|---|---|
| | | | of Previous |
| Gender (n) | Ethnicity | Age (years) | Endoscopies (n) |
| Male = 13 | Caucasian = 19 | 13.04 | 2.26 |
| Female = 9 | (1 not enrolled) | (SD +/− 2.7) | (SD +/− 1.15) |
| (1 not enrolled) | Native American = 1 | | |
| | Hispanic = 2 | | |

TABLE 2
Duration of TNE

| | | | Average Number |
|---|---|---|---|
| | | | of Previous |
| Gender (n) | Ethnicity | Age (years) | Endoscopies (n) |
| Male = 13 | Caucasian = 19 | 13.04 | 2.26 |
| Female = 9 | (1 not enrolled) | (SD +/− 2.7) | (SD +/− 1.15) |
| (1 not enrolled) | Native American = 1 | | |
| | Hispanic = 2 | | |

TABLE 3
Adverse Events

| | Total Number of Subjects |
|---|---|
| Self Reported Symptom | Reporting Symptom |
| Nausea | 4 |
| Choking/Gagging | 12 |
| Sore Throat | 10 |
| Vomiting | 2 |
| Chest Pain | 2 |
| Abdominal Pain | 1 |
| Other | 4 (2 reporting nose discomfort; |
| | 2 reporting slightly sore throat) |
| No Significant Symptoms | 7 |

TABLE 4
Satisfaction with the Procedure

| Instrument/Question | Instrument Score | |
|---|---|---|
| mGHAA-9 Score (Max 45 Points) | 43.19 | (SD +/− 2.6) |
| Qualitative Satisfaction Instrument | Total Subject of 21 total (n) | |
| Child: Satisfaction with TNE | 17 | (81%) |
| Parent: Satisfaction with TNE | 19 | (90.5%) |
| Parent/Child Satisfied with Sedated EGD | 17 | (81%) |
| Parent/Child Concerned with Sedated EGD | 13 | (61.9%) |
| Child: Willing to Repeat TNE | 16 | (76.2%) |
| Parent: Willing to Repeat TNE | 21 | (100%) |

| | |
|---|---|
| Child: Prefer to Repeat TNE | 11 (52.4%) - 4 prefer |
| | neither EGD or TNE |
| Parent: Prefer to Repeat TNE | 18 (85.7%) - 1 prefer |
| | neither EGD or TNE |
| Parent: Qualitative Advantages of TNE | 13/21 - No anesthesia |
| | 11/21 - Faster procedure |
| | and recovery |
| | 6/21 - Parental presence |
| | in the procedure room |
| | 4/21 - Lower cost |

TABLE 5
TNE Findings

| TNE Findings | Total Specimens (n) |
|---|---|
| Visually Normal | 11 |
| Slight Furrowing | 2 |
| Furrowing | 8 |
| | (1 with exudates) |
| Normal Biopsy Abnormality | 12 |
| Eosinophils > 15 hpf | 5 |
| Eosinophils > 15 hpf | 4 |

| | | Average Epitheleal | |
|---|---|---|---|
| Biopsy Forceps | Sample Size | Surface Area (mm²) | P-value |
| EGD 2.8 mm* | n = 11 | 0.38 (SD 0.14) | P = 0.308 |
| biopsy forceps | | | |
| TNE 1.2 mm | | 0.33 (SD 0.09) | |
| biopsy forceps | | | |
| EGD 2.8 mm* | n = 9 | 0.52 (SD 0.19) | P = 0.496 |
| biopsy forceps | | | |
| TNE 2.0 mm | | 0.50 (SD 0.15) | |
| biopsy forceps | | | |

What is claimed is:

1. An endoscope for assessment of the esophageal mucosa in children comprising:
a flexible endoscope shaft having a first end, a second end, a length of about 1.0 meter to about 1.1 meter, an outer diameter of between about 3.0 mm to about 4.0 mm and having an inner channel lumen of about 1.5 mm to about 2.5 mm in diameter, the lumen extending substantially the length of the shaft, wherein the shaft is configured to facilitate irrigation and suction at the second end of the shaft;
a handle disposed at first end of the shaft, the handle including a single or dual control to adjust the disposition of the second end of the shaft thereby enabling four-way tip deflection;
an image sensor at the second end of the shaft to facilitate imaging at the distal end of the endoscope when the endoscope is inserted within a cavity of a subject; and
a light source disposed at the second end of the shaft to illuminate the area surrounding the distal end of the shaft.

2. The endoscope according to claim 1 further comprising a foot pedal or hand control to actuate suction or irrigation of the endoscope.

3. The endoscope according to claim 1 further comprising a control integral to the handle to actuate suction or irrigation of the endoscope.

4. The endoscope according to claim 1 further comprising a camera to facilitate visualization within the cavity of the subject.

5. The endoscope according to claim 1 wherein the image sensor is a sensor selected from the group consisting of a charge-coupled device (CCD) sensor, a complementary metal-oxide-semiconductor (CMOS) sensor, an N-type metal-oxide-semiconductor (NMOS) sensor and a high definition video chip.

6. The endoscope according to claim 1 further comprising a scope shaft stiffening component to selectively reduce the flexibility of the scope shaft.

7. The endoscope according to claim 6 wherein the scope shaft stiffening component is adapted to facilitate the use of the endoscope in aerodigestive medicine.

8. The endoscope according to claim 1 wherein the lumen has an opening at the distal-most end of the second end of the shaft.

9. An endoscope for transnasal endoscopy in children comprising:
a flexible endoscope shaft having a first end and a second end, and has a diameter dimensioned for insertion into the nasal cavity of a child, a length of about 0.8 meters to 1.2 meters, and has an inner channel lumen configured to receive an elongate surgical instrument, the lumen extending substantially the length of the shaft, wherein the shaft is configured to facilitate irrigation and suction at the second end of the shaft;
a handle disposed at first end of the shaft, the handle including a single or dual control to adjust the disposition of the second end of the shaft thereby enabling four-way tip deflection;
an image sensor at the second end of the shaft to facilitate imaging at the distal end of the endoscope when the endoscope is inserted within the nasal cavity of a subject; and
a light source disposed at the second end of the shaft to illuminate the area surrounding the distal end of the shaft.

10. The endoscope according to claim 9 further comprising a foot pedal or hand control to actuate suction or irrigation of the endoscope.

11. The endoscope according to claim 9 further comprising a control integral to the handle to actuate suction or irrigation of the endoscope.

12. The endoscope according to claim 9 further comprising a camera to facilitate visualization within the cavity of the subject.

13. The endoscope according to claim 9 wherein the image sensor is a sensor selected from the group consisting of a charge-coupled device (CCD) chip, a complementary metal-oxide-semiconductor (CMOS) or N-type metal-oxide-semiconductor (NMOS) and a high definition video chip.

14. The endoscope according to claim 9 further comprising a scope stiffening component to selectively reduce the flexibility of the scope.

15. The endoscope according to claim 14 wherein the scope stiffening component is adapted to facilitate the use of the endoscope in aerodigestive medicine.

16. The endoscope according to claim 9 wherein the lumen has an opening at the distal-most end of the second end of the shaft.

17. An endoscope for transnasal endoscopy in children comprising:
a flexible endoscope shaft having a first end, a second end, a shaft diameter dimensioned for insertion into the nasal cavity of a child, a shaft length adapted to facilitate insertion to the esophageal mucosa of a child, and having an inner channel lumen configured to receive an elongate surgical instrument, the lumen extending substantially the length of the shaft, wherein the shaft is configured to facilitate irrigation and suction at the second end of the shaft;
a handle disposed at first end of the shaft, the handle including a single or dual control to adjust the disposition of the second end of the shaft thereby enabling four-way tip deflection;
an image sensor at the second end of the shaft to facilitate imaging at the distal end of the endoscope when the endoscope is inserted within the nasal cavity of a subject; and
a light source disposed at the second end of the shaft to illuminate the area surrounding the distal end of the shaft.

18. The endoscope according to claim 17 further comprising a foot pedal or hand control to actuate suction or irrigation of the endoscope.

19. The endoscope according to claim 17 wherein the image sensor is a sensor selected from the group consisting of a charge-coupled device (CCD) chip, a complementary metal-oxide-semiconductor (CMOS) or N-type metal-oxide-semiconductor (NMOS) and a high definition video chip.

20. The endoscope according to claim 17 further comprising a scope stiffening component to selectively reduce the flexibility of the scope.
